(19) United States
(12) Patent Application Publication
(10) Pub. No.: US 2008/0233624 A1
(43) Pub. Date: Sep. 25, 2008

(54) Apparatus and method for biohydrogen production

(76) Inventors: Lynne Elaine Macaskie, Oxford (GB); Mark Derek Redwood, Nottingham (GB)

Correspondence Address: BERLINER & ASSOCIATES, 555 WEST FIFTH STREET, 31ST STREET, LOS ANGELES, CA, 90013, US

(21) Appl. No.: 11/881,959

(22) Filed: Jul. 30, 2007

(30) Foreign Application Priority Data
Mar. 23, 2007 (GB) 0705583.3

(51) Int. Cl. C12P 3/00; C12M 1/00

(52) U.S. Cl. 435/168; 435/297.2

(57) ABSTRACT

An apparatus for biohydrogen production comprises a cell with an anion-selective membrane dividing the cell into first and second compartments. In use, the first compartment is placed into fluid communication with a bacterial fermentation culture and the second compartment is placed into fluid communication with a photoheterotrophic bacterial culture. Application of a potential difference to the cell allows organic acids produced by the bacterial fermentation culture to cross the membrane and be supplied to the photoheterotrophic bacterial culture. Regulation of the current through the cell controls the quantity of ammonium transferred with the organic acids.

The present invention relates to the production of hydrogen using bacteria (biohydrogen production). More specifically, it relates to an apparatus and a method for biohydrogen production by fermentation of sugars by bacteria such as Escherichia coli, and photofermentation of the resulting organic acids by photoheterotrophic bacteria such as Rhodobacter sphaeroides. The present invention further relates to a method to improve biohydrogen production, and the uses of electric current in such methods to control ammonium transport and to improve biohydrogen production.

Biohydrogen is anticipated to play an important role in the future hydrogen economy, as it can be produced from readily available renewable substrates. Sugars are promising substrates for biological H₂ production, being readily and renewably available and potentially giving a high yield of H₂ (Equation I).

C₆H₁₂O+6H₂O→12H₂+6CO₂ (I)

The stoichiometric yield of 12 mol H₂ per mol hexose represents the ultimate target for biohydrogen production. No single organism is capable of performing the conversion with this efficiency. In fact, the thermodynamic maximum yield for dark fermentation is 4 mol/mol (the Thauer limit) as illustrated in Equation II:

### Fermentation:

C₆H₁₂O₆+2H₂O→4H₂+2CH₃COOH+2CO₂ (II)

In order to improve the H₂ production efficiency of the process, therefore, it is necessary to further convert the organic acids of Equation II, theoretically producing a further 8 mol of H₂:

### Photofermentation:

2CH₃COOH+4H₂O→8H₂+4CO₂ (III)

Equations II and III describe an ideal situation in which all carbon substrate is processed along the appropriate pathways and none is diverted to the formation of biomass or alternative metabolites. In practice, fermentation will produce a range of organic compounds, according to the precise fermentation conditions used. In order to maximise the efficiency of the process, it is necessary that as many as possible of these organic compounds can be converted to produce hydrogen gas.

Similarly, although the fermentation of Equation II is shown acting on a simple hexose, in practice a sugar feed solution may also contain more complex carbohydrates. In order to maximise the efficiency of the process, it is necessary that different hexoses and more complex carbohydrates can be converted to produce hydrogen gas.

According to a first aspect of the present invention, there is provided an apparatus for biohydrogen production, comprising a cell having an anion-selective membrane dividing the cell into first and second compartments, the first compartment having a cathode, and the second compartment having an anode, wherein the first compartment is in fluid communication with a bacterial fermentation culture, and the second compartment is in fluid communication with a photoheterotrophic bacterial culture.

In one embodiment, the apparatus additionally comprises a first reactor for fermentation, in fluid communication with the first compartment of the cell. This reactor may store the majority of the bacterial fermentation culture, with culture medium from the first reactor being pumped to the first compartment of the cell and then returning to the first reactor. This allows the volume of culture medium to be varied, whilst maintaining the same design of cell. It also reduces the proportion of time for which any given bacterium is subject to the effects of the electric field within the cell; exposure to electric field is thought to reduce the viability of cell cultures.

In one embodiment, the apparatus additionally comprises a second reactor for photoheterotrophy, in communication with the second compartment of the cell. This reactor may store the majority of the photoheterotrophic bacterial culture, with culture medium from the second compartment of the cell being pumped to the second reactor, and then returning to the second compartment of the cell. As above, this allows for flexibility in the volume of culture medium used, and reduces the proportion of time for which the individual bacteria of this culture are exposed to the electric field within the cell.

According to a second aspect of the present invention there is provided a method for biohydrogen production, comprising:
a) providing an apparatus comprising a cell having an anion-selective membrane dividing the cell into first and second compartments, the first compartment having a cathode, and the second compartment having an anode, wherein the first compartment is in fluid communication with a bacterial fermentation culture, and the second compartment is in fluid communication with a photoheterotrophic bacterial culture;
b) supplying the bacterial fermentation culture with an aqueous solution of at least one fermentable carbohydrate, such that the bacterial fermentation culture ferments the fermentable carbohydrate and produces at least one organic acid;
c) supplying the first compartment of the cell with culture medium from the bacterial fermentation culture;
d) applying a potential difference between the anode and the cathode to cause the at least one organic acid to cross the anion-selective membrane from the first compartment of the cell to the second compartment of the cell;
e) supplying the photoheterotrophic bacterial culture with fluid from the second compartment of the cell, such that the photoheterotrophic culture ferments the at least one organic acid and produces hydrogen gas;
f) collecting hydrogen gas produced by the photoheterotrophic bacterial culture.

This process involves a first fermentation stage, in which fermentable sugar is converted into organic acids. Although some hydrogen may be produced by this stage, the accumulation of other fermentation products (such as organic acids) can reduce or halt fermentation, even in an excess of substrate. Furthermore, the presence of the organic acids in the residual medium would present difficulties when disposing of the medium. According to the process of the present invention, therefore, organic acids produced by fermentation in the first stage are extracted from the fermentation medium (by means of the anion-selective membrane) and passed to a second photoheterotrophic stage in which the organic acids are further converted to H₂. Utilization of fermentation end-products for further H₂ production in a second stage increases the economic potential of the process by improving the H₂ yield and reducing the organic content of the final waste.

In one embodiment, the bacterial fermentation culture also produces hydrogen.

In one embodiment, the fermentation culture medium after step b) comprises dissolved ammonium. Such ammonium ions may be produced by the bacterial fermentation culture as part of the fermentation process. Alternatively, the ammonium may have been present in the initial fermentable carbohydrate solution, or may have been formed by the bacterial fermentation culture by reduction of nitrate or nitrite present in the fermentable carbohydrate solution.

Although the culture medium from the first (“fermentation”) stage of the process is able, following pH adjustment, to directly support growth of photoheterotrophic bacteria, the present inventors have surprisingly found that when the fermentation culture medium after step b) comprises ammonium, little or no hydrogen production occurs in the absence of the apparatus and method of the current invention. It is thought that nitrogenase (the enzyme thought to be responsible for H₂ production in the second, heterotrophic phase) is inhibited by the ammonium. However, by utilising the anion-selective membrane of the present invention, the majority of the ammonium is separated from the organic acids required for the second stage photoheterotrophic fermentation, allowing H₂ production to take place, as required.

One example of a membrane-equipped cell suitable for use in the present invention is described in International (PCT) Patent Application WO 2004/046351. The cell described in this document has an anion-selective membrane (Neosepta ACM) separating the Bio-Reaction Chamber (equivalent to the first compartment of the present invention) from the Product Concentrate Chamber (equivalent to the second compartment). The teaching of that document is incorporated herein by reference.

According to a third aspect of the present invention, there is provided a method for biohydrogen production, comprising:
a) providing an apparatus comprising a cell having an anion-selective membrane dividing the cell into first and second compartments, the first compartment having a cathode, and the second compartment having an anode, wherein the first compartment is in fluid communication with a bacterial fermentation culture, and the second compartment is in fluid communication with a photoheterotrophic bacterial culture;
b) supplying the bacterial fermentation culture with an aqueous solution of at least one fermentable carbohydrate, such that the bacterial fermentation culture ferments the fermentable carbohydrate and produces at least one organic acid, and the resulting culture medium comprises dissolved ammonium;
c) supplying the first compartment of the cell with culture medium from the bacterial fermentation culture;
d) applying a potential difference between the anode and the cathode to cause an electric current to flow between the anode and the cathode, and thereby to cause the at least one organic acid to cross the anion-selective membrane from the first compartment of the cell to the second compartment of the cell;
e) regulating the electric current flowing between the anode and the cathode such that ammonium is transferred across the anion-selective membrane from the first cell compartment to the second cell compartment;
f) supplying the photoheterotrophic bacterial culture with culture medium from the second compartment of the cell, such that the photoheterotrophic culture ferments the at least one organic acid and produces hydrogen gas;
g) collecting hydrogen gas produced by the photoheterotrophic bacterial culture.

Although phototrophic H₂ production by anoxygenic photosynthetic bacteria (APB) is thought to be inhibited by ammonium, it is known that small quantities of a nitrogen source such as the ammonium ion are in fact essential for the growth of the bacteria. The inventors have surprisingly found that, by ensuring that the current density across the membrane is maintained within certain limits, it is possible to cause small quantities of ammonium to cross the membrane, in spite of the opposing potential difference. Thus, by regulation of the current density, it is possible to regulate the supply of ammonium ion to the bacteria in the photobioreactor and hence to maximise H₂ production.

In general, the rate of ammonium transfer decreases exponentially with increasing current density, as can be seen from FIG. 3. Although generally it is desirable to maximise the current density and thereby maximise the transfer of organic acids from the first to the second compartment of the cell, in the method of this aspect of the present invention, the current is regulated to allow ammonium transport.

The precise ranges of current density will vary according to the system, but can be readily determined by the skilled man by measuring ammonium transfer for different current densities. Ammonium transfer can be measured by any appropriate method, such as for example by using the cell of the present invention without the bacterial cell cultures. The first cell compartment may then be filled with a solution of ammonium sulphate, the second cell compartment filled with an ammonium-free solution, and a known electric current passed through the cell. Samples taken from the second cell compartment at regular intervals may be tested for ammonium concentration (for example, using the indophenol blue method, Nessler method or, for real-time measurement, an ammonium probe)

In one embodiment, the electric current flowing through the cell is varied between a maximum level at which substantially no ammonium is transferred from the first cell compartment to the second cell compartment across the anion-selective membrane, to a minimum level at which ammonium is so transferred.

The quantity of ammonium transferred from the first cell compartment to the second cell compartment in step e) should be sufficient to enable detectable growth of bacteria of the photoheterotrophic bacterial culture. Such growth may be measured by any appropriate means, such as for example by measuring optical density.

In one embodiment, the bacterial fermentation culture also produces hydrogen.

A further benefit of the methods according to the present invention is that the electrokinetic cell acts as a microfiltration unit, retaining the bacterial fermentation culture in the first stage of the process, while extracting water to maintain a constant culture volume, despite the continuous addition of feed and titrant to the culture. Water transport across the membrane occurs as a result of electrodialysis. In a single stage process, this effect would normally be considered a disadvantage, since it limits the concentration of extracted product (e.g. the organic acids) which is achievable. However, in the present invention, this effect helps to carry the acids to the second stage.

According to a fourth aspect of the current invention, there is provided the use of an electrical current applied through the anion-selective membrane of the apparatus of the third aspect of the present invention, in order to regulate the transfer of ammonium from the first cell compartment to the second cell compartment through the membrane.

In one embodiment, the use comprises varying the magnitude of the electric current.

According to a fifth aspect of the present invention, there is provided the use of direct electrical current to improve gaseous hydrogen production by dark fermenting bacteria capable of anaerobic fermentation of sugars to produce organic acids and hydrogen, the use comprising applying the current to a bacterial fermentation culture.

In one embodiment, the bacterial fermentation culture comprises E. coli.

The following optional embodiments apply to all aspects of the present invention.

As used herein, the term “bacterial fermentation culture” refers to a bacterial culture which comprises any bacterial strain capable of anaerobic fermentation of sugars to produce organic acids. In one embodiment, the bacterial fermentation culture comprises at least one bacterial strain capable of anaerobically fermenting sugars to produce organic acids and hydrogen. In a further embodiment, the bacterial fermentation culture comprises E. coli. In a further embodiment still, the bacterial fermentation culture comprises the hydrogen-overproducing E. coli strain HD701 (M. Sauter et al, Mol Microbiol 1992, vol. 6, p. 1523-32). Alternatively or additionally, the properties of the bacterial culture may be altered in any manner known to the skilled man, including by genetic engineering, and may for example include genetic modifications known to increase the hydrogen production of bacterial cultures.

As used herein, the term “photoheterotrophic bacterial culture” refers to a bacterial culture which comprises any bacterial strain capable of anaerobic fermentation of organic acids under the action of light to produce hydrogen gas. Such bacteria may be known as anoxygenic photosynthetic bacteria (APB). In one embodiment, the photoheterotrophic bacterial culture comprises R. sphaeroides.

According to one embodiment of the present invention, hydrogen production of the photoheterotrophic bacterial culture is inhibited by presence of ammonium. This is true for all wild-type anoxygenic photosynthetic bacteria although some genetically engineered strains are known in which this behaviour is suppressed or removed.

According to one embodiment of the present invention, hydrogen gas is also collected from the cathode of the electrokinetic cell.

The inventions of the present application will now be illustrated by the following specific examples with reference to the Figures, in which:

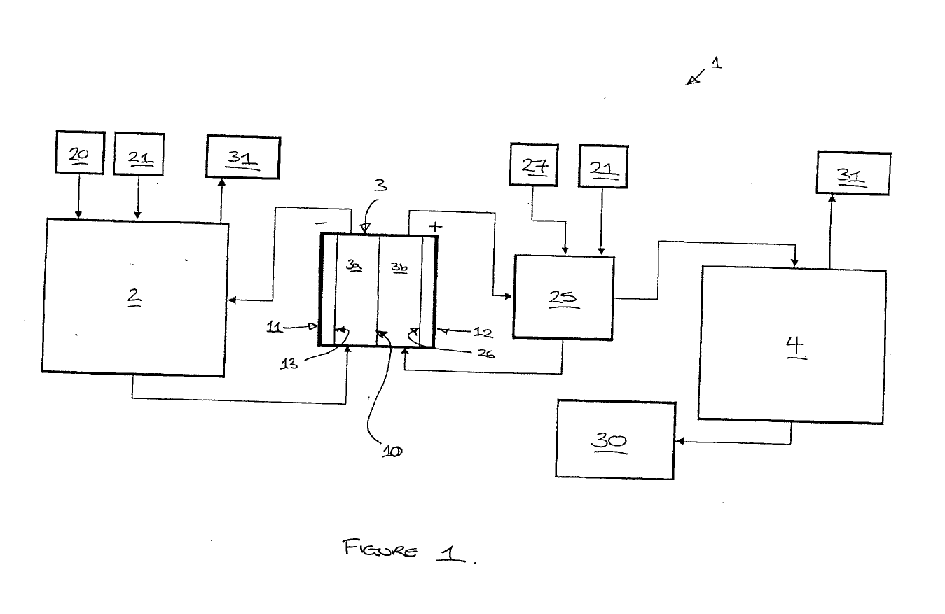

FIG. 1 represents a schematic diagram of an apparatus suitable for use in the present invention;

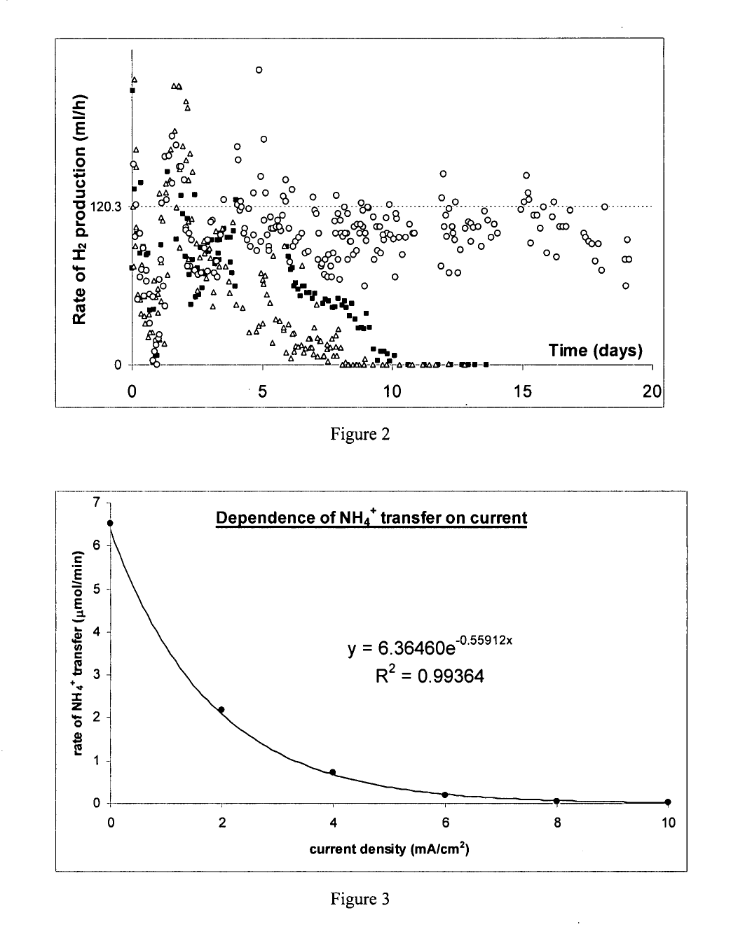

FIG. 2 shows the results of a method according to the second aspect of the present invention;

FIG. 3 shows the effect of current density in the electrokinetic cell on ammonium transport across an anion-selective membrane (area 200 cm²); and

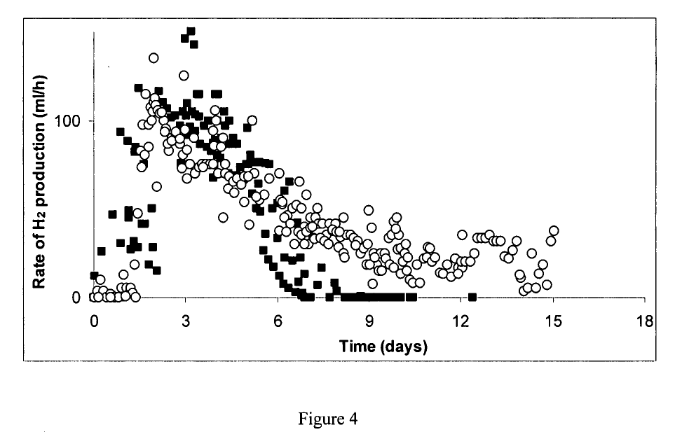

FIG. 4 shows the results of a method according to the second aspect of the present invention.

### EXAMPLE 1

### Apparatus

Referring to FIG. 1, the biohydrogen production system 1 comprises a dark fermentation vessel 2 (6 litre, Electrolab, UK), a cell 3, and a photobioreactor 4. Cell 3 is divided into first and second compartments 3a and 3b by means of an anion-selective membrane 10 (Neosepta AHA). The first compartment 3a is fitted with a stainless steel cathode 11, whilst the second compartment 3b is fitted with a platinum-coated anode 12; both electrodes are connected to a power supply (not shown). Culture medium from the fermentation vessel 2 is pumped through the first cell compartment 3a and then returned to the vessel 2; within the first cell compartment 3a the culture medium is protected from the cathode 11 by a bipolar membrane 13 (BP-1E). Within the bipolar membrane, the cathode 11 is immersed in a circulating solution of 0.5M sodium sulphate (not shown).

Pumps are connected to supply the fermentation vessel 2 with sugar feed 20 and pH titrants 21 as necessary.

Fluid is circulated through the second cell compartment 3b from a permeate vessel 25; within the second cell compartment 3b, the fluid is protected from the anode 12 by a cation-selective membrane 26. Within the cation-selective membrane 26, the anode 12 is immersed in a circulating solution of 0.5M sodium sulphate (not shown).

Pumps are connected to supply the permeate vessel 25 with basal medium 27 and pH titrants 21 as necessary. Basal medium 27 supplies the photobioreactor 4 with all growth requirements (including a nitrogen source) except for organic acids, which are acquired from the second cell compartment 3b.

Fluid from the permeate vessel 25 (including that which has circulated through the second cell compartment 3b) is supplied to the photobioreactor 4. Excess fluid from the photobioreactor 4 is separately discharged to waste 30. Hydrogen gas 31 is collected from both the fermentation vessel 2 and the photobioreactor 4.

Pre-Culture of E. coli

The H₂-overproducing strain Escherichia coli HD701 was kindly provided by Professor A. Bock (Lehrstuhl für Mikrobiologie, Munich, Germany) and cultured aerobically on nutrient broth (Oxoid) supplemented with 0.5% sodium formate (w/w) (1 litre, 16 h, 200 rpm, 0.002% inoculum v/v). A standard temperature of 30° C. was upheld for all growth stages and fermentations.

### Feeding Regime for Dark Fermentation

The fermentation vessel 2 was autoclaved with 2.8 litres of aqueous fermentation medium as shown in Table 1.

TABLE 1
Fermentation medium (2.8 L, pH 5.5)

| | | | |
|---|---|---|---|
| Na₂SO₄ | 42.6 | g | Sterilised as 2.8 L |
| K₂HPO₄ | 10.456 | g | |
| KH₂PO₄ | 0.204 | g | |
| (NH₄)₂SO₄ | 0.198 | g | |
| 1M MgSO₄ | 6 | ml | Sterilised |
| trace elements solution* | 9 | ml | individually and |
| 2M glucose | 30 | ml | added separately |

The fermentation vessel 2 (in fluid communication with the 1^{st} cell compartment 3a) contained initially 2.8 litres of complete fermentation medium (above). E. coli cells (2 litres) were harvested from the pre-growth medium by centrifugation (4435×g, 20° C., 10 min) resuspended in 200 ml saline (NaCl 0.85% (w/w), pH 7.0) and inoculated into the fermentation vessel 2. Thus, the initial glucose concentration was 20 mM. The complete culture was purged with argon for 30 minutes before the commencement of gas measurement. Electrodialysis (400 mA, ca. 4V) was activated on the medium 1 hour prior to inoculation.

The permeate vessel 25 (in fluid communication with the 2^{nd} cell compartment 3b) contained initially 1 litre of basal medium (described below).

The addition of feed solution 1 to the fermentation vessel 2 commenced 24 h following the initiation of dark fermentation (100 mL/day, 0.6 M glucose, 0.015 M (NH₄)₂SO₄). This point coincided with the continuous addition of basal medium 27 (1 litre/day) to the permeate vessel 25 to generate organic-acid enriched medium. At the same point the contents of the permeate vessel 25 were continuously supplied to the photobioreactor 4 (1 litre/day).

Pre-Culture of R. sphaeroides

R. sphaeroides was pre-cultured in 15 mL water-jacketed vials filled with sterile succinate medium (Hoekema et al., International Journal of Hydrogen Energy, 2002, vol. 27(11-12), p. 1331-1333) under tungsten illumination (30° C., 72 h).

### Photobioreactor Specifications

Photofermentation was carried out in a cylindrical glass photobioreactor 4 (internal diameter, 105 mm). The illuminated surface area was 0.107 m² and the average intensity of photosynthetically active radiation at the culture surface was 117.4 μE/m²/s, which was provided by 3 clear tungsten filament bulbs arranged externally along the length of the photobioreactor. The cylinder was surrounded in a reflective tube (diameter 35 cm). The culture (3.0±0.5 litre) was stirred using a magnetic stirrer and follower (1200 rpm) located at the base of the photobioreactor. A temperature of 30.0±0.2° C. was maintained using a submerged cooling coil.

### Feeding/Dilution Regime for Photofermentation

The photobioreactor 4 was autoclaved and filled with 3 litres of mixed organic acid growth medium (below), inoculated with 30 ml pre-culture (above) and sparged with argon for 30 min. After growing for 72 h, the contents of the permeate vessel 25 were continuously added to the photobioreactor 4 (1 litre/day) and the contents of the photobioreactor 4 were continuously withdrawn into the outflow vessel 30. This point coincided with the continuous addition of feed solution 1 to the fermentation vessel 2 (100 ml/day).

TABLE 2
Mixed organic acid growth medium for R. sphaeroides

| | | |
|---|---|---|
| Acetate | 16 | mM |
| Succinate | 14 | mM |
| Lactate | 8 | mM |
| Butyrate | 5 | mM |
| KH₂PO₄ | 1.466 | g/litre |
| K₂HP0₄ | 1.732 | g/litre |
| Yeast extract | 1.00 | g/litre |
| Vitamins solution* | 2 | ml |
| Macroelements solution* | 50 | ml |
| Microelements solution* | 10 | ml |

*as described by Hoekema et al., International Journal of Hydrogen Energy, 2002, vol. 27(11-12), p. 1331-1333

The composition of basal medium 27 was identical to that of the mixed organic acid growth medium except for the absence of organic acids (acetate, succinate, butyrate and lactate).

FIG. 2 demonstrates the effect of the invention on hydrogen production in the fermentation vessel 2. Control experiments (closed squares) were carried out using a simple fermentation vessel without the electrokinetic cell of the present invention. As can be seen, hydrogen production dropped to zero after approximately 10 days. A second control (open triangles) was performed using the configuration shown in FIG. 1 except that the anion selective membrane 10 was replaced with an inactive membrane (the membrane no longer transported organic acids because it was aged or fouled). Thus, under the influence of direct current without the extraction of organic acids, hydrogen production had ceased by approximately 8 days. However, in the experiment involving electrodialysis (open circles) according to the present invention, the rate of hydrogen production remained high even after approximately 20 days. The dotted line drawn at 120.3 mL/h indicates a 100% yield—the H₂ production rate given a yield of 2 mol H₂/mol glucose and a glucose load of 60 mmol/day.

FIG. 4 demonstrates the effect of the invention on hydrogen production in the photobioreactor vessel 4. Control experiments (closed squares) were performed using the configuration shown in FIG. 1 except that the anion selective membrane 10 was replaced with an inactive membrane (the membrane no longer transported organic acids because it was aged or fouled). There is a peak in hydrogen production at around day 3 as the bacteria are allowed to grow in the growth medium. After day 3 the medium is diluted, as described above and hydrogen production tails off. There is very little or no hydrogen production from day 8. However, in the experiment involving electrodialysis (open circles) the hydrogen production continues beyond day 8. This can be attributed to organic acid transport across the membrane 10 and its subsequent photofermentation.

### Ammonium Transport

Ammonium transport across the anion-selective membrane was measured and the results are shown in FIG. 3. It can be seen that the ammonium flux over 200 cm² membrane varies from 0-6.5 μmol/min for current densities in the range 10-0 mA/cm², adhering closely to an exponential function. Thus, at higher current densities, when organic acid transport is most efficient, the level of ammonium transport is very low. At lower current densities, the ammonium transport level increases up to a maximum.

### EXAMPLE 2

The method of Example 1 was repeated, except that in this example the nitrogen requirements of the photofermentation were provided entirely through the transfer of ammonium ion from the first stage dark fermentation via the electrodialysis cell, rather than being added separately via the basal medium.

The apparatus shown in FIG. 1 was modified by inclusion of a feedback turbidostat circuit in which a decrease in the optical density of the photoheterotrophic bacterial culture in the photobioreactor 4 prompts a period of decreased current applied to the cell 3, causing increased ammonium ion transfer through the anion-selective membrane 10. This in turn produces a period of growth, causing the turbidity of the photoheterotrophic bacterial culture to increase.

In addition, growth supplements (trace elements and vitamins) of negligible volume were supplied directly to the photobioreactor 4, in place of the supply of basal medium to the permeate vessel 25. The direct addition of growth supplements to the photobioreactor 4 means that the bacteria are not dependent on a supply from the permeate vessel 25 and so the fluid flow rate can be adjusted as required. A minimum flow rate is required in order to transport ammonium ion and organic acid from cell compartment 3b, via the permeate vessel 25 to the photobioreactor 4 but since the photobioreactor is of finite size, any excess fluid must be discharged as waste 30.

Turbidity measurements were taken to determine the required quantity of growth and hence the required quantity of ammonium. Current was then reduced to supply it over a short period, after which the current was returned to the original high setting.

In accordance with Example 1 the photofermentation was required to process an estimated 250 mmol carbon/day as a mixture of organic acids. This requires ca. 3.55 g R. sphaeroides biomass (dry weight), which is 8.73% N (w/w) (published value). The supply of ammonium ion necessary to support this culture is dependent upon the dilution rate of the photofermentation culture. The apparatus of this experiment was designed to minimise the dilution rate of the photobioreactor, and hence minimise the necessary ammonium supply. The dilution rate is an uncontrolled variable equal to the sum of water transport via the electrokinetic cell from the dark fermentation and the addition of pH titrant to the permeate chamber. Growth supplements added to the photobioreactor are of negligible volume. A typical dilution rate would be 150 ml/day (Hydraulic retention time, HRT=20 days), necessitating a daily nitrogen supply of 15.5 mg/day, equal to 0.763 μmol ammonium ion/min.

### Nitrogen Supply Regime

To maximise current for organic acid transfer, the ammonium ion transfer was conducted in short periods of low current. For example a current of 0.1 mA/cm² would be used to supply the daily requirement of 1.10 mmol ammonium ion in 3.04 hours, the remainder of the period being dedicated to organic acid transport employing a higher current.

1. An apparatus for biohydrogen production, comprising a cell having an anion-selective membrane dividing the cell into first and second compartments, the first compartment having a cathode, and the second compartment having an anode, wherein in use the first compartment is in fluid communication with a bacterial fermentation culture and the second compartment is in fluid communication with a photoheterotrophic bacterial culture.

2. The apparatus of claim 1, additionally comprising a first reactor for fermentation, in fluid communication with the first compartment of the cell.

3. The apparatus of claim 1, additionally comprising a second reactor for photoheterotrophy, in communication with the second compartment of the cell.

4. A method for biohydrogen production, comprising:
(a) providing an apparatus comprising a cell having an anion-selective membrane dividing the cell into first and second compartments, the first compartment having a cathode, and the second compartment having an anode;
(b) bringing the first compartment of the cell into fluid communication with a bacterial fermentation culture, and the second compartment into fluid communication with a photoheterotrophic bacterial culture;
(c) supplying the bacterial fermentation culture with an aqueous solution of at least one fermentable carbohydrate, such that the bacterial fermentation culture ferments the fermentable carbohydrate and produces at least one organic acid;
(d) supplying the first compartment of the cell with culture medium from the bacterial fermentation culture;
(e) applying a potential difference between the anode and the cathode to cause the at least one organic acid to cross the anion-selective membrane from the first compartment of the cell to the second compartment of the cell;
(f) supplying the photoheterotrophic bacterial culture with fluid from the second compartment of the cell, such that the photoheterotrophic culture ferments the at least one organic acid and produces hydrogen gas; and
(g) collecting hydrogen gas produced by the photoheterotrophic bacterial culture.

5. The method of claim 4, wherein the bacterial fermentation culture also produces hydrogen in step (c), and step (g) further comprises collecting the hydrogen gas produced by the bacterial fermentation culture.

6. The method of claim 4, wherein the culture medium of step (d) comprises dissolved ammonium.

7. A method for biohydrogen production, comprising:
(a) providing an apparatus comprising a cell having an anion-selective membrane dividing the cell into first and second compartments, the first compartment having a cathode, and the second compartment having an anode;
(b) bringing the first compartment into fluid communication with a bacterial fermentation culture, and the second compartment into fluid communication with a photoheterotrophic bacterial culture;
(c) supplying the bacterial fermentation culture with an aqueous solution of at least one fermentable carbohydrate, such that the bacterial fermentation culture ferments the fermentable carbohydrate and produces at least one organic acid, and the resulting culture medium comprises dissolved ammonium;
(d) supplying the first compartment of the cell with culture medium from the bacterial fermentation culture;
(e) applying a potential difference between the anode and the cathode to cause an electric current to flow between the anode and the cathode, and thereby to cause the at least one organic acid to cross the anion-selective membrane from the first compartment of the cell to the second compartment of the cell;
(f) regulating the electric current flowing between the anode and the cathode such that ammonium is transferred across the anion-selective membrane from the first cell compartment to the second cell compartment;
(g) supplying the photoheterotrophic bacterial culture with culture medium from the second compartment of the cell, such that the photoheterotrophic culture ferments the at least one organic acid and produces hydrogen gas; and
(h) collecting hydrogen gas produced by the photoheterotrophic bacterial culture.

8. The method of claim 7, wherein the electric current flowing through the cell is varied between a maximum level at which substantially no ammonium is transferred from the first cell compartment to the second cell compartment across the anion-selective membrane, and a minimum level at which ammonium is so transferred.

9. The method of claim 7, wherein the bacterial fermentation culture also produces hydrogen, and step (h) further comprises collecting the hydrogen gas produced by the bacterial fermentation culture.

10. The use of an electrical current applied through the anion-selective membrane of an apparatus of claim 1, in order to regulate the transfer of ammonium from the first cell compartment to the second cell compartment through the membrane.

11. The use of claim 10, wherein the use comprises varying the magnitude of the electric current.

12. The use of direct electrical current to improve gaseous hydrogen production by dark fermenting bacteria capable of anaerobic fermentation of sugars to produce organic acids and hydrogen, the use comprising applying the current to a bacterial fermentation culture.
